(19) United States
(12) Patent Application Publication
(10) Pub. No.: US 2019/0282541 A1
(43) Pub. Date: Sep. 19, 2019

(54) USE OF ERIBULIN AND HISTONE DEACETYLASE INHIBITORS IN THE TREATMENT OF CANCER

(71) Applicant: EISAI R&D MANAGEMENT CO., LTD., Tokyo (JP)

(72) Inventors: Bruce A. LITTLEFIELD, Andover, MA (US); Gary HENDLER, Hatfield (GB)

(21) Appl. No.: 16/318,198

(22) PCT Filed: Jul. 20, 2017
(86) PCT No.: PCT/JP2017/026212

§ 371 Date: Jan. 16, 2019

Related U.S. Application Data
(60) Provisional application No. 62/364,440, filed on Jul. 20, 2016.

(51) Int. Cl. A61K 31/357; A61K 31/4406; A61K 31/565; A61P 35/04

(52) U.S. Cl. CPC A61K 31/357; A61K 31/4406; A61K 45/06; A61P 35/04; A61K 31/565

(57) ABSTRACT

The invention features methods for treating and preventing cancer (e.g., a hormone sensitive cancer) in a patient in need thereof by administering eribulin (e.g., eribulin mesylate) in combination with a histone deacetylase (HDAC) inhibitor.

### BACKGROUND OF THE INVENTION

Cancer is characterized by the uncontrolled growth of a particular type of cell. It begins in a tissue containing such a cell and, if the cancer has not spread to any additional tissues at the time of diagnosis, may be treated by, for example, surgery, radiation, or another type of localized therapy. However, when there is evidence that cancer has metastasized from its tissue of origin, different approaches to treatment are typically used. Indeed, because it is not possible to determine with certainty the extent of metastasis, systemic approaches to therapy are usually undertaken when any evidence of spread is detected. These approaches can involve the administration of chemotherapeutic drugs that interfere with the growth of rapidly dividing cells, such as cancer cells. Other approaches involve the use of immunotherapy, in which an immune response against cancerous cells in a subject is elicited or enhanced.

Halichondrin B is a structurally complex, macrocyclic compound that was originally isolated from the marine sponge Halichondria okadai, and subsequently was found in Axinella sp., Phakellia carteri, and Lissodendoryx sp. A total synthesis of halichondrin B was published in 1992 (Aicher et al., J. Am. Chem. Soc. 114:3162-3164, 1992). Halichondrin B has been shown to inhibit tubulin polymerization, microtubule assembly, beta^{s}-tubulin crosslinking, GTP and vinblastine binding to tubulin, and tubulin-dependent GTP hydrolysis in vitro. This molecule has also been shown to have anti-cancer properties in vitro and in vivo. Halichondrin B analogs having anti-cancer activities are described in U.S. Pat. No. 6,214,865 B1.

Eribulin is a synthetic analog of halichondrin 8. Eribulin is also known as ER-086526, and has been assigned CAS number 253128-41-5 and US NCI designation number NSC-707389. The mesylate salt of eribulin (eribulin mesylate, which is marketed under the trade name HALAVEN® and is also known as E7389) is approved in certain jurisdictions for the treatment of patients with breast cancer and for the treatment of patients with liposarcoma. In the U.S., for example, HALAVEN® is approved for the treatment of patients with breast cancer who have previously received at least two chemotherapeutic regimens for the treatment of metastatic disease that should have included an anthracycline and a faxane in either the adjuvant or metastatic setting, and for second line liposarcoma treatment

The chemical name for eribulin mesylate is 11,15:18,21:24,28-triepoxy-7,9-ethano-12,15-methano-9H, 15H-furo[3,2-i]furo [2′,3′,5,6]pyrano[4,3-b][1,4]dioxacyclopentacosin-5(4H)-one, 2-[(2 S)-3-amino-2-hydroxypropyl]hexacosahydro-3-methoxy-26-methyl-20,27-bis(methylene)-, (2 R,3 R,3 aS,7 R,BaS,9 S,10 aR,11 S,12 R, 13 aR,13 bS,15 S,18 S,21 S,24 S,26 R,28 R,29 aS)-methanesulfonate (salt), and it can be depicted as set forth below.

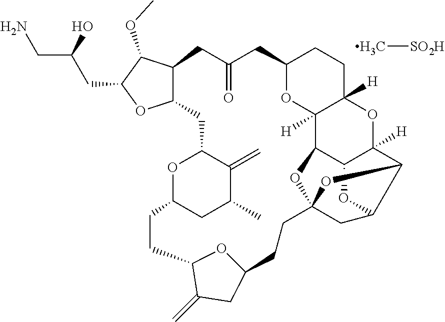

Cancer is typically characterized by changes in the DNA sequences of certain genes relating to the control of cell growth and proliferation. In addition to sequence changes, cancer may be associated with epigenetic changes in these genes (e.g., modifications in histone acetylation and/or DNA methylation) which, although not causing any sequence changes, result in altered gene expression. In one example, disruption of histone acetyltransferase and histone deacetylase (HDAC) has been associated with a variety of different types of cancers, thus leading to these enzymes as being targets for anti-cancer drug development. There are four classes of HDACs, with three of them (I, II, and IV) comprising eleven zinc-dependent metalloproteins Entinostat (MS-275) is a Class I HDAC inhibitor. The chemical name of entinostat is pyridin-3-yl methyl N-[[4-[(2-aminophenyl)carbarnoyl]phenyl]methyl carbamate, and it can be depicted as set forth below.

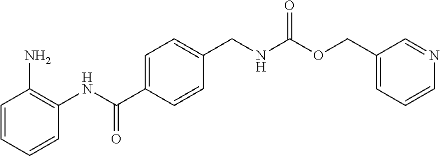 [SMILES: Nc1ccccc1NC(=O)c1ccc(CNC(=O)OCc2cccnc2)cc1]

### SUMMARY OF THE INVENTION

The present invention provides methods of treating and preventing cancer (e,g., hormone responsive cancer) by administration of eribulin, or a pharmaceutically acceptable salt thereof (e.g., eribulin mesylate), and an HDAC inhibitor (e.g., entinostat). When the term “eribulin” is used herein, it should be considered as indicating eribulin or a pharmaceutically acceptable salt thereof (such as eribulin mesylate), unless the context indicates otherwise.

The invention provides methods for treating a subject (e.g., a human) having or at risk of developing cancer. The methods include administering to the subject (a) eribulin, or a pharmaceutically acceptable salt thereof (e.g., eribulin mesylate), and (b) a histone deacetylase (HDAC) inhibitor (e.g., a hydroxamic acid derivative, a carboxylic acid derivative, a benzamide derivative, a cyclic peptide, or an epoxyketone). In specific examples, the HDAC inhibitor is selected from the group consisting of trichostatin A, vorinostat, panobinostat, belinostat, givinostat, practinostat, quisinostat, abexinostat, CHR-3996, AR-42, valproate, butyrate, entinostat, entinostat polymorph B, mocetinostat, chidamide, apicidin, romidepsin, and trapoxins.

The methods of the invention can optionally consist of administering (a) eribulin mesylate and (b) the HDAC inhibitor to the subject, or consist of administering (a) eribulin mesylate and (b) entinostat to the subject. In various embodiments, (a) and (b) are administered substantially simultaneously; (a) is administered first, followed by administration of (b); (b) is administered first, followed by administration of (a); (a) and (b) are administered substantially simultaneously, followed by administration of (a); or (a) and (b) are administered substantially simultaneously, followed by administration of (b).

The subject treated according to the methods of the invention can optionally be diagnosed with cancer, in treatment for cancer, or in post-therapy recovery from cancer. The cancer treated according to the methods of the invention can optionally be a primary tumor, locally advanced, or metastatic. Furthermore, in other examples, the cancer can optionally be hormone responsive.

In various examples, the cancer is selected from the group consisting of breast cancer, sarcomas, endometrial cancer, ovarian cancer, prostate cancer, leukemia, lymphoma, lung cancer, neuroendocrine tumors, pheochromocytoma, and thyroid cancer.

In further examples, the cancer is a breast cancer selected from the group consisting of triple-negative breast cancer, triple-positive breast cancer, HER2-negative breast cancer, HER2-positive breast cancer; estrogen receptor-positive breast cancer, estrogen receptor-negative breast cancer, progesterone receptor-positive breast cancer. progesterone receptor-negative breast cancer, ductal carcinoma in situ (DCIS), invasive ductal carcinoma, invasive lobular carcinoma, inflammatory breast cancer, Paget disease of the nipple, and phyliodes tumor.

In additional examples, the cancer is a sarcoma selected from the group consisting of angiosarcoma, hemangiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leiomyosarcoma, and neurofibrosarcoma.

In yet further examples, the cancer is selected from the group consisting of stomach cancer, colon cancer, liver cancer, renal cancer, colorectal cancer, pancreatic cancer, cervical cancer, anal cancer, vulvar cancer, penile cancer, vaginal cancer, testicular cancer, pelvic cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer. melanoma, a cancer of the central nervous system, a cancer of the respiratory system, and a cancer of the urinary system.

In further examples, the cancer is selected from the group consisting of B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), erythroleukemia, basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastic carcinoma, adrenocortical carcinoma, cholangiocarcinoma, Merkel cell carcinoma, ductal carcinoma in situ (DCIS), invasive ductal carcinoma, hepatoblastoma, medulloblastoma, nephroblastoma, neuroblastoma, pancreatoblastoma, pleuropulmonary blastoma, retinoblastoma, glioblastoma multiforme, Hodgkin's lymphoma, non-Hodgkin's lymphoma, Burkitt lymphoma, follicular lymphoma, thymorne, multiple myeloma, plasmacytoma, localized myeloma, extramedullary myeloma, superficial spreading melanoma, nodular melanoma, lentigno maligna melanoma, acral lentiginous melanoma, amelanotic melanoma, ganglioneuroma, Pacinian neuroma, acoustic neuroma, astrocytoma, oligoastrocyloma, ependymoma, brainstem glioma, optic nerve glioma, oligoastrocytoma, pheochromocytoma, meningioma, malignant mesothelioma, and a virally induced cancer.

The subject treated according to the methods of the invention may be a human patient, including adult patients and pediatric patients.

In various embodiments, the eribulin or the pharmaceutically acceptable salt thereof (e.g., eribulin mesylate) is administered by intravenous infusion for, e.g., about 1 to about 20 minutes, or about 2 to about 5 minutes. In additional embodiments, the eribulin or the pharmaceutically acceptable salt thereof can be administered in an amount in the range of, e.g., about 0.1 mg/m² to about 20 mg/m², or about 1.1 mg/m² or 1.4 mg/m2.

In further embodiments, the HDAC inhibitor can he administered orally in an amount ranging from, for example. 0.5-30 mg/day on, e.g., a weekly or biweekly basis. In one example, the HDAC inhibitor is entinostat, which is administered in an amount of, e.g., about 4-10 mg/m².

Treatment according to the methods of the invention optionally (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

Also provided by the invention are methods for decreasing the size of a tumor in a subject, e.g., as described herein. These methods include administering to the subject (a) eribulin, or a pharmaceutically acceptable salt thereof (e.g., eribulin mesylate), and (b) an HDAC Inhibitor (e.g., entinostat), for example, as described herein.

The methods of the invention, such as those described above and elsewhere herein, can further optionally include administering to the subject one or more additional therapeutic agents, which optionally are selected from anti-hormonal agents (e.g., fulvestrant, tamoxifen, toremifene, or aromatase inhibitors), immunomodulatory agents (e.g., antibodies or vaccines), chemotherapeutic/antitumor agents, antibacterial agents, anti-emetics, and anti-inflammatory agents.

The invention also includes kits for use in treating cancer or decreasing tumor size in a subject, e.g., as described herein. The kits can include (a) eribulin, or a pharmaceutically acceptable salt thereof (e.g., eribulin mesylate), and (b) an HDAC inhibitor (e.g., entinostat), optionally in dosage form.

The invention also includes pharmaceutical compositions as described herein for use in the treatment of cancer, as described herein, as well as use of the recited agents (i.e., eribulin or a pharmaceutically acceptable salt thereof (e.g., eribulin mesylate) and an HDAC inhibitor (e.g., entinostat) in the treatment of cancer, as described herein. Thus, the invention includes eribufin, or a pharmaceutically acceptable salt thereof, for use in a method for treating a subject having or at risk of developing cancer, the method including administering to the subject (a) eribulin, or pharmaceutically acceptable salt thereof, and (b) a histone deacetylase (HDAC) inhibitor. The invention also includes eribulin, or a pharmaceutically acceptable salt thereof, for use in a method of making a medicament for treating a subject having or at risk of developing cancer, the method including administering to the subject (a) eribulin, or pharmaceutically acceptable salt thereof, and (b) a histone deacetylase (HDAC) inhibitor,

The methods of the invention provide improved approaches for treating cancer. For example, the combination treatment methods described herein can be used to obtain synergistic effects in which, for example, the effects are greater than the sum of the effects of the drugs administered individually, as can be determined by those of skill in the art. Additive effects, which are also beneficial, can also be achieved.

Other features and advantages of the invention will be apparent from the following detailed description and the claims.

### DETAILED DESCRIPTION

The invention provides methods for the treatment or prevention of cancer (e.g., hormone responsive cancer) involving administration of eribulin or a pharmaceutically acceptable salt thereof (e.g., eribulin mesylate) in combination with a histone deacetylase (HDAC) inhibitor (e.g., entinostat).

Treatment of cancer according to the methods of the invention, can (i) reduce the number of cancer cells; (ii) reduce tumor volume; (iii) increase tumor regression rate; (iv) reduce or slow cancer cell infiltration into peripheral organs; (v) reduce or slow tumor metastasis; (vi) reduce or inhibit tumor growth; (vii) prevent or delay occurrence and/or recurrence of the cancer and/or extend disease- or tumor-free survival time; (viii) increase overall survival time; (ix) reduce the frequency of treatment; and/or (x) relieve one or more of symptoms associated with the cancer.

### Pharmaceutical Compositions, Dosage, and Methods

Pharmaceutical compositions including eribulin and/or an HDAC inhibitor can be prepared using standard methods known in the art, or can be obtained from commercial sources. Typically, eribulin and the HDAC inhibitor used in the invention are included within separate pharmaceutical compositions but they can, optionally, be included within a single composition. Eribulin is typically provided in liquid form, for intravenous administration, while the HDAC inhibitor may optionally be formulated, for example, for oral or intravenous formulation, depending upon the inhibitor selected.

Pharmaceutical compositions used in the invention can be prepared by, for example, mixing or dissolving the active ingredient(s), having the desired degree of purity, in a physiologically acceptable diluent, carrier, excipient, or stabilizer (see, e.g., Remington's Pharmaceutical Sciences (22nd edition), ed. A. Germaro, 2012, Lippincott, Williams & Wilkins, Philadelphia, Pa.). Acceptable diluents include water and saline, optionally including buffers such as phosphate, citrate, or other organic acids, antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

In preparing compositions for oral dosage form, any of the usual pharmaceutical media can be employed, for example, water, glycols, oils, alcohols, flavoring agents, preservatives, coloring agents. In addition, carriers such as starches, sugars, microcrystalline cellulose, diluents, granulating agents, lubricants, binders, disintegrating agents, and the like can be used in the case of oral solid preparations such as, for example, powders, capsules, and tablets.

Optionally, the formulations of the invention contain a pharmaceutically acceptable preservative. In some embodiments the preservative concentration ranges from 0.1 to 2.0%, typically v/v. Suitable preservatives include those known in the pharmaceutical arts, such as benzyl alcohol, phenol, m-cresol, methylparaben, and propylparaben. Furthermore, the eribulin and/or the HDAC inhibitor formulations can optionally include a pharmaceutically acceptable salt, such as sodium chloride at, for example, about physiological concentrations. Thus, in one example, eribulin (e.g., eribulin mesylate) is formulated in 0.9% Sodium Chloride Injection (USP).

The formulations noted above (and others) can be used for administration of the drugs. Thus, the drugs can be administered by mutes including intravenous, intra-tumoral, peri-tumoral, intra-arterial, intra-dermal, intra-vesical, ophthalmic, intramuscular, intradermal, intraperitoneal, pulmonary, subcutaneous, and transcutaneous routes. Other routes can also be used including, for example, oral, transmucosal, transdermal, inhalation, intravaginal, and rectal administration routes.

The dosage of eribulin and the HDAC inhibitors described herein administered can differ markedly depending on the type of target disease, the choice of delivery method, as well as the age, sex, and weight of the patient, the severity of the symptoms, along with other factors. The dosage to use can be determined by those of skill in the art based on factors such as these. Eribulin and examples of HDAC inhibitors that can be used in the methods of the invention, as well as administration regimens, are described further below.

### Eribulin

Methods for the synthesis of eribulin are described, for example, in U.S. Pat. No. 6,214,865, U.S. Pat. Nos. 7,982,060, 8,350,067; and 8,093,410, each of which is incorporated herein by reference. As noted above, eribulin mesylate is available commercially and is marketed as HALAVEN®.

Eribulin can optionally be used in the present invention in salt forms. There are no particular limitations as to the salt used, whether inorganic acid salt or organic acid salt. For example, the salt can be selected from mesylic acid salt (e.g., eribulin mesylate), hydrochloric acid salt, sulfuric acid salt, citrate, hydrobromic acid salt, hydroiodine acid salt, nitric acid salt, bisulfate, phosphoric acid salt, super phosphoric acid salt, isonicotinic acid salt, acetic add salt, lactic acid salt, salicic acid salt, tartaric acid salt, pantotenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate), and so on. Moreover, it is acceptable to use salt of aluminum, calcium, lithium, magnesium, sodium, zinc, and diethanolamine.

The daily dosage of eribulin (e.g., eribulin mesylate) can be in the range of, e.g., 0.001 mg/m² to about 100 mg/m² (e.g., in the range of about 0.1 mg/m² to about 50 mg/m² or in the range of about 0.7 mg/m² to about 1.5 mg/m², or in any single amount within these ranges (e.g., 1.4 mg/m² or 1.1 mg/m²)). Eribulin can be administered as a single dose once per day, week, month, or year, or more than one dose of eribulin can be administered per day, week, month, or year. For example, in one administration protocol, eribulin can be administered once on days 1 and 8 of a 21-day cycle. More specifically, a recommended dose of eribulin mesylate is 1.4 mg/m² administered intravenously over 2 to 5 minutes on days 1 and 8 of a 21-day cycle. A recommended dose of eribulin mesylate in patients with mild hepatic impairment (Child-Pugh A) is 1.1 mg/m² administered intravenously over 2 to 5 minutes on days 1 and 8 of a 21-day cycle, while a recommended dose of eribulin mesylate in patients with moderate hepatic impairment (Child-Pugh B) is 0.7 mg/m² administered intravenously over 2 to 5 minutes on days 1 and 8 of a 21-day cycle. Further, a recommended dose of eribulin mesylate in patients with moderate renal impairment (creatinine clearance of 30-50 mL/min) is 1.1 mg/m² administered intravenously over 2 to 5 minutes on days 1 and 6 of a 21-day cycle. These or other lower doses of eribulin mesylate can optionally be used in the context of combination treatment, according to the methods of the present invention.

### HDAC Inhibitors

HDAC inhibitors that can be used in the invention can be specific for any HDAC within Class 1, II, 111, or IV, or can be active against one, more than one, or all of these classes (e.g., an HDAC inhibitor can be active against HDAC Classes I, II, and IV). HDAC inhibitors can be classified based on their specificity or, alternatively, based on their chemical structures. For example, HDAC inhibitors that can be used in the invention can optionally fall within one of the following classes, based on their chemical structures: hydroxamic acid derivatives (e.g., trichostatin A, vorinostat [SAHA], panobinostat [LBH-589], belinostat [PXD101; Beleodaq], givinostat [ITF2357], practinostat [SB939], quisinostat [JNJ-26481585], abexinostat [PC1-24781], CHR-3996, and AR-42), carboxylic acid derivatives (e.g., valproate and butyrate), benzamide derivatives (e.g., entinostat [MS275], entinostat polymorph B, mocetinostat, and chidamide [CS055/HBI-8000], cyclic peptides (e.g., apicidin arid romidepsin [Istodax; Depsipeptide]), and epoxyketones (e.g., trapoxins).

In one specific example, the HDAC inhibitor used in the invention is entinostat, which is a highly selected Class I HDAC inhibitor. Methods for the synthesis of entinostat are described. For example, in U.S. Pat. No. 6,794,392; WO 01/12193; and U.S. Pat. No. 7,973,166 (polymorph B), each of which is incorporated herein by reference. Entinostat can be administered as a single dose once per day, week, month, or year, or more than one dose of entinostat can be administered per day, week, month, or year, optionally by the oral route. For example, in one administration protocol, entinostat can be administered (e.g., by the oral route) once on each of days 1 and 8 of a 21- or 28-day cycle. In other examples, entinostat is administered on a daily, semi-weekly, weekly, bi-weekly, or monthly basis. Each dose of entinostat can be, for example, 0.5-50 mg (e,g., 1, 2.5, 5, 7.5, 10, 12.5, 15, 20, 25, 30, 35, 40, or 45 mg), administered optionally by the oral route. In other examples, the dosage is 4-10 mg/m², e.g., 4, 5, 6, 7, 8, 9, or 10 mg/m² per day by the oral route.

### Combination Administration Regimens

As noted above, according to the methods of the invention, eribulin (e.g., eribulin mesylate) and an HDAC inhibitor (e.g., entinostat; also see list above) are administered in combination. In some embodiments, eribulin (e.g., eribulin mesylate) arid the HDAC inhibitor (e.g., entinostat; also see list above) are administered substantially simultaneously. In some embodiments, eribulin (e.g., eribulin mesylate) and the HDAC inhibitor (e.g., entinostat; also see list above) are administered separately, e.g., eribulin is administered first, followed by administration of the HDAC inhibitor; or HDAC inhibitor is administered first, followed by administration of the eribulin. In some embodiments, eribulin (e.g., eribulin mesylate) and the HDAC inhibitor (e,g., entinostat; also see list above) are administered substantially simultaneously, followed by administration of eribulin or the HDAC inhibitor. In some embodiments, eribulin (e,g, eribulin mesylate) or the HDAC inhibitor (e.g., entinostat; also see list above) is administered first, followed by administration of eribulin arid the HDAC inhibitor substantially simultaneously. The administrations can begin on the same day or treatment using one agent can start, e.g., 1, 2, 3, 4, 5, or 6 weeks before treatment the other, as can be determined to be appropriate by those of skill in the art.

In addition to eribulin and one or more HDAC inhibitor (e.g., entinostat; also see list above), the methods of the present invention can also include the administration of one or more additional therapeutic agents. Among these agents, anti-hormonal agents (e.g., fulvestrant, tamoxifen, toremifene, or aromatase inhibitors), immunomodulatory agents (e.g., antibodies or vaccines), chemotherapeutic/antitumor agents, antibacterial agents, anti-emetics, and anti-inflammatory agents are suitable. In other instances, eribulin (e.g., eribulin mesylate) and one or more HDAC inhibitor (e.g., entinostat; also see list above) can be used in a treatment regimen as the sole therapeutic (e.g., sole anti-cancer) agents. Thus, the methods of the invention can consist of administration of (a) eribulin or a pharmaceutically acceptable salt thereof (e.g., eribulin mesylate), and (b) an HDAC inhibitor (e.g., entinostat, also see list above)

### Cancers

The methods of the invention can be used to treat (including, e.g., delay progression) or prevent cancer (e.g., a hormone responsive cancer) in a subject (e.g., a human patient) and/or to decrease tumor size. A hormone responsive cancer is characterized by cancer cells that are typically stimulated to proliferate in response to a hormone (for example, a steroid hormone [e.g., estrogen, progesterone, testosterone, or a corticosteroid] or thyroid hormone). The subject can be diagnosed with cancer (e.g., a hormone responsive cancer), at risk for developing cancer, in treatment for cancer, or in post-therapy recovery from cancer. Further, the methods can be used to treat or prevent metastases and/or recurrence. The treatment can be chemotherapeutic alone, although treatment in combination with a surgical procedure to remove or reduce the size of a tumor (e.g., neo-adjuvant treatment), radiation therapy, anti-hormonal, immunotherapy, and/or ablation therapy is also included in the invention.

The cancer may be a primary tumor, locally advanced, or metastatic, and optionally may be hormone responsive, as noted above. In various examples, the cancer is selected from the group consisting of breast cancer, endometrial cancer, ovarian cancer, prostate cancer, leukemia, lymphoma, lung cancer (e.g., small cell lung cancer), neuroendocrine tumors (e.g., neuroendocrine tumor of the pancreas), pheochromocytoma, and thyroid cancer, with each of these examples optionally being hormone responsive. In other examples, the cancer can be selected from the group consisting of stomach cancer, colon cancer, liver cancer, renal cancer, colorectal cancer, pancreatic cancer, cervical cancer, anal cancer, vulvar cancer, penile cancer, vaginal cancer, testicular cancer, pelvic cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer (e.g., melanoma), a cancer of the central nervous system, a cancer of the respiratory system, and a cancer of the urinary system.

In more detail, cancers that can be treated or prevented according to the methods of the invention include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leiomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastic carcinoma, adrenocortical carcinoma, cholangiocarcinoma, Merkel cell carcinoma, ductal carcinoma in situ (DCIS), and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, Burkitt lymphoma, and follicular lymphoma), thymoma, myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

The methods of the invention can be used to treat or prevent breast cancer (e.g., triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor). Of particular note is estrogen receptor-positive breast cancer, as well as triple-negative breast cancer.

Patients that can be treated according to the methods of the invention include adults (e.g., people older than 18 or 21 years of age), as well as pediatric patients (e,g., patients up to and including the age of 18 or 21 years of age), who have cancer, e.g., a cancer type listed herein. In regard to pediatric patients, specific examples of cancers that can be treated include, e.g., sarcomas and leukemias, such as those listed above.

### Kits

The invention also provides kits that include a container with eribulin (e.g., eribulin mesylate) and/or a container with an HDAC inhibitor described herein (e.g., entinostat; also see above). The eribulin and/or the HDAC inhibitor in such kits can be provided in amounts sufficient to treat cancer (e.g., a hormone responsive cancer; see, the lists set forth above) in a patient in need thereof (e.g., amounts sufficient for a single administration or for multiple administrations). The kits can thus include multiple containers that each include effective amounts of single-dose eribulin and/or the HDAC inhibitor pharmaceutical composition(s). Optionally, instruments and/or devices necessary for administering the pharmaceutical composition(s) can also be included in the kits. Furthermore, the kits can include additional components, such as instructions or administration schedules. For treating a patient with cancer (e.g., a hormone responsive cancer) with the eribulin and/or the HDAC inhibitor described herein.

The present invention is illustrated by the following examples, which are in no way intended to be limiting of the invention.

### EXAMPLES

The growth inhibitory activities of two test agents, eribulin and entinostat, were determined alone and in combination against 12 human breast cancer cell lines (Table 1). The human breast tumor cell lines were selected based on their profile characteristics of being either human epidermal growth factor receptor 2 negative (Her2′) and estrogen receptor positive (ER⁺) or Triple Negative (Her2⁻, ER⁻, and progesterone receptor negative [PR⁻]). Activities of individual agents were determined by curve-fitting the concentration of agent that inhibits cell growth by 50% compared to the control cells in a four parameter-logistic equation, whereas agent combinations were determined by the method of (Chou and Talelay Advances in Enzyme Regulation 22:27-55, 1984), and analyzed with CalcuSyn software. Fa, the fraction of affected drug targets at a given drug concentration, was calculated from the fractional decrease. Each agent was tested individually to determine the median-effect dose, Dm, (or EC₅₀, the 50% effect concentration). For combination treatments, drug solutions were mixed at ratios providing approximately equipotent concentrations based upon relative Dm, and this mixture was serially diluted to yield test concentrations. The Combination Index (CI), computed from ratios of the concentrations of mixed and unmixed drugs that produce the same Fa, indicates whether inhibition is additive, synergistic, or antagonistic. In whole-cell assays, inhibitor interactions are complex (mixed), rather than mutually exclusive (purely competitive) or nonexclusive (purely non-competitive), CI values for both types of inhibition are reported to define the range of possible values.

TABLE 1
Cell Lines Summary

| Cell line | Histotype | Profile Status |
|---|---|---|
| MCF-7 | Human Mammary Gland | Her2⁻ and ER⁺ |
| | Adenocarcinoma | |
| T-47D | Human Mammary Ductal | Her2⁻ and ER⁺ |
| | Carcinoma from Metastatic Site: | |
| | Pleural Effusion | |
| ZR-75-1 | Human Mammary Ductal | Her2⁻ and ER⁺ |
| | Carcinoma | |
| MDA-MB-134VI | Human Breast Ductal Carcinoma | Her2⁻ and ER⁺ |
| MDA-MB-175VII | Human Mammary Gland Ductal | Her2⁻ and ER⁺ |
| | Carcinoma | |
| MDA-MB-415 | Human Mammary gland/breast; | Her2⁻ and ER⁺ |
| | derived from metastatic site | |
| MDA-MB-231 | Human Mammary Gland | Triple Negative |
| | Adenocarcinoma | |
| HCC70 | Human Breast Ductal Carcinoma | Triple Negative |
| HCC1806 | Human Squamous Carcinoma | Triple Negative |
| BT-549 | Human Breast Carcinoma | Triple Negative |
| Hs578t | Human Breast Carcinoma | Triple Negative |
| MDA-MB-436 | Human Adenocarcinoma | Triple Negative |
| | Derived from Metastatic Site | |

We first determined the antiproliferative activity of the study agents alone against the panel of human tumor cell lines using a wide range of agent concentrations, and then analyzed duplicate samples over a ten point dose response range (Experiment 1). Next, we determined the antiproliferative activity of the study agents alone against the panel of human tumor cell lines using a narrow range of agent concentrations, and analyzed duplicate samples over a ten or twenty point dose response range (Experiment 2). Finally, in Experiment 3, we determined the antiproliferative activity of the study agents in combination against the panel of human tumor cell lines. The IC₅₀ values of the agents determined were used to determine appropriate drug ratios and concentration ranges for a combination study based on the constant ratio design of Chou-Talalay (Advances in Enzyme Regulation 22:27-55, 1984). We further analyzed quadruplicate samples over a seven point dose response range.

### Materials and Methods

### Cell Culture

MCF-7 human breast cancer cells were cultured in RPMI 1640 medium supplemented with 10% fetal bovine serum (FBS), 2 mM glutamine, 10 mM 4-(2-hydroxyethyl)-1-piperazineethanesulfonic acid (HEPES), 0.075% sodium bicarbonate, 100 units/mL sodium penicillin G, 100 μg/mL streptomycin sulfate and 25 μg/mL gentamicin. T-47D human breast cancer cells were cultured in RPMI 1640 medium supplemented with 10% FES. 4.5 g/L glucose. 2 mM glutamine. 10 mM HEPES, 0.2 units/mL Insulin, 1 mM sodium pyruvate, 100 units/mL sodium penicillin G, 100 streptomycin sulfate and 25 μg/mL gentamicin, ZR-75-1 human breast cancer cells were cultured in RPMI 1640 medium supplemented with 10% FBS, 4.5 g/L. glucose, 2 mM glutamine, 10 meat HEPES, 1 mM sodium pyruvate, 100 units/mL sodium penicillin G, 100 μg/mL streptomycin sulfate and 25 μg/mL gentamicin. MDA-MB-134-VI human breast cancer cells were cultured in Leilbovitz's L-15 medium supplemented with 20% FBS, 2 mM glutamine, 100 units/mL sodium penicillin G, 100 μg/mL streptomycin sulfate and 25 μg/mL gentamicin. MDA-MB-175-VII human breast cancer cells were cultured in Leilbovitz's L-15 medium supplemented with 10% FBS. 2 mM glutamine, 10 mM HEPES, 100 units/mL sodium penicillin G, 100 μg/mL streptomycin sulfate and 25 μg/mL gentamicin. MDA-MB-415 human mammary glanclibreast cancer cells were cultured in Leilbovitz's L-15 medium supplemented with 15% FES, 10 μg/mL human insulin, 10 μg/mnL glutathione, 2 mM glutamine, 100 units/mL sodium penicillin G, 100 μg/mL streptomycin sulfate, and 25 μg/mL gentamicin. MDA-MB231, HCC1806, BT-549, and Hs578t human breast cancer cells were cultured in RPMI 1640 medium supplemented with 10% FBS, 2 mM glutamine, 100 units/mL sodium penicillin G, 100 μg/mL streptomycin sulfate and 25 μg/mL gentamicin. HCC70 human breast cancer cells were cultured in RPMI1640 medium supplemented with 10% FBS, 2 mM glutamine, 4.5 g/L glucose, 10 mM HEPES, 0.075% sodium bicarbonate, 1 mM sodium pyruvate, 100 units/mL sodium penicillin 0, 100 μg/mL streptomycin sulfate and 25 μg/mL gentamicin. MDA-MB-436 human adenocarcinoma derived cancer cells were cultured in Leilbovitz's L-15 medium supplemented with 10% FBS, 10 μg/mL bovine insulin, 16 μg/mL glutathione, 2 mM glutamine, 100 units/mL sodium penicillin G, 100 μg/mL streptomycin sulfate and 25 μg/mL gentamicin. The tumor cells were cultured in tissue culture flasks in a humidified incubator at 37° C., in an atmosphere of 5% CO₂ and 95% air; except for the MDA-MB-134-VI, MDA-MB-175-VII, MDA-MB-415, and MDA-MB-436, these cells were cultured at 37° C. in 100% air and no additional CO₂.

### Therapeutic Agents

Eribulin was supplied as a stock solution (10 mM), and was stored protected from light at −80° C. On Day 1 of the study, the stock was thawed and a 1000× stock (1 mM) was prepared using 10 μL of the stock solution diluted in 90 μL of DMSO. The 1000× stock was used to prepare the 10× (10 μM) drug prep solution in 1% DMSO in media.

Entinostat was obtained from SelleckChem (Catalog No. 81116) and supplied at a 10 mM stock solution in DMSO, and stored at −80° C. The 10 mM stock served as the 1000× stock and a 10× stock was prepared by diluting the 10 mM stock diluted in 1% DMSO in media to a concentration of 100 μM.

### Proliferation Determination

The selected human tumor cells were seeded at 2,000-5,000 cells/well in a clear polystyrene 96-well microculture plate (Corning® Costar® 96-well flat bottom plate, Cat. No. 3997) in a total volume of 90 μL per well. After 24 hours of incubation in a humidified incubator at 37° C. with 5% CO₂ and 95% air (except for the MDA-MB-134-VI, MDA-MB-175-VII, MDA-MB-415, and MDA-MB-436, these cells were cultured at 37° C. in 100% air, no additional CO₂), 10 μL of the 10× drug prep was added. For Experiment 1, 10 μL of the 10× eribulin drug prep solution was added to 90 μL of cells plated in media for a final concentration of 1 μM at the top concentration diluted 1:4 for a total of 10 dilutions. For entinostat, 10 μL of the 10× drug prep solution was added to 90 μL of cells plated in media for a final concentration of 10 μM at the top concentration diluted 1:4 for a total of 10 dilutions. For Experiment 2, narrow range IC₅₀ value determination of single agents, 10 μL of the 10× drug prep solution was added to 90 μL of cells plated in media for final concentrations as described in Table 2. For MDA-MB-436, MDA-MB-134VI, MDA-MB-175VII, and MDA-MB-415 the media was replaced and fresh drug was added at 72 hours after cell plating. After 72 hours (144 hours for MDA-MB-436, MDA-MB-134VI, MDA-MB-175VII, and MDA-MB-415) of culture, the plated cells and Cell Titer-Glo® (Promega #07571) reagents were brought to room temperature and allowed to equilibrate for 30 minutes. One hundred (100) μL of the Cell Titer-Glo® reagent was added to each well. The plate was shaken for two minutes to induce lysis and then left to equilibrate for ten minutes to stabilize the luminescent signal. The medium/Cell Titer-Glo® reagent was transferred to a white polystyrene 96-well microculture plate (Corning® Costar® 96-well flat bottom plate, Cat. No. 3917) before determining luminescence on the Tecan GENios microplate. All determined IC₅₀ values were rounded to show up to two decimals. Percent inhibition of cell growth was calculated relative to untreated control wells. All tests were performed in duplicate at each concentration level. The IC₅₀ value for the test agents was estimated using Prism 6.05 by curve-fitting the data using the following four parameter-logistic equation:

$$Y = \frac{Top - Bottom}{1 + {( \frac{X}{{IC}_{50}} )}^{n}} + Bottom$$

where Top is the maximal % of control luminescence, Bottom is the minimal % of control luminescence at the highest agent concentration, Y is the % of control luminescence, X is the agent concentration, IC₅₀ is the concentration of agent that inhibits cell growth by 50% compared to the control cells, and n is the slope of the curve.

TABLE 2
Concentration of agents for narrow range IC₅₀ value determination

| | | Top |
|---|---|---|
| Cell line | Compound | Concentration |
| MCF-7 | eribulin | 8 nM |
| | entinostat | 10 μM |
| T-47D | eribulin | 8 nM |
| | entinostat | 20 μM |
| ZR-75-1 | eribulin | 20 μM |
| | entinostat | 40 μM |
| MDA-MB-134VI | eribulin | 20 μM |
| | entinostat | 40 μM |
| MDA-MB-175VII | eribulin | 20 μM |
| | entinostat | 40 μM |
| MDA-MB-415 | eribulin | 40 nM |
| | entinostat | 10 μM |
| MDA-MB-231 | eribulin | 8 nM |
| | entinostat | 10 μM |
| HCC70 | eribulin | 20 μM |
| | entinostat | 40 μM |
| HCC1806 | eribulin | 8 nM |
| | entinostat | 10 μM |
| BT-549 | eribulin | 8 nM |
| | entinostat | 20 μM |
| Hs578t | eribulin | 8 nM |
| | entinostat | 20 μM |
| MDA-MB-436 | eribulin | 20 μM |
| | entinostat | 40 μM |

### IC₅₀ Value Determination of Single Agents in Combination

The IC₅₀ values of the agents determined under Experiment 2 were used to determine appropriate drug ratios and concentration ranges for a combination study based on the constant ratio design of Chou-Talalay (Advances in Enzyme Regulation 22:27-55, 1984). The cancer cells were seeded at 2,000-5,000 cells/well in a clear polystyrene 96-well microculture plate (Corning® Costar® 96-well flat bottom plate, Cat. No. 3997) in a total volume of 90 μL/well. After 24 hours of incubation in a humidified incubator at 37 with 5% CO₂ and 95% air (except for the MDA-MB-134-VI, MDA-MB-175-VII, MDA-MB-415, and MDA-MB-436, these cells were cultured at 37° C. in 100% air, no additional CO₂), 10 μL of 10× serially diluted test agents in growth medium were added to each well in quadruplicate. For MDA-MB-436, MDA-MB-134VI, MDA-MB-175VII, and MDA-MB-415 the media was replaced and fresh drug was added at 72 hours after cell plating. The cells (quadruplicate wells) were incubated with a combination of compounds based upon IC₅₀ determined in Experiments 1 and 2 (Tables 3 and 4). Drug ratios were equivalent to the ratio of respective IC₅₀ of agents being combined. For a combination response of near additive, drug concentrations bracketed the sum of one half of the respective IC₅₀ with serial dilutions selected based upon the inhibition curves of the agents being combined (1.25 fold dilutions) with a total of 7 drug concentrations. After 72 hours (144 hours for MDA-MB-436, MDA-MB-134VI, MDA-MB-175VII, and MDA-MB-415) of drug incubation, the cell number was determined with the Cell Titer assay as described above. The combination data analysis was performed using the CalcuSyn Software 2.0 developed by Chou and Talalay (distributed by BioSoft). To determine whether the test agents produce synergistic, additive, or antagonistic effects, a scale was used for interpretation of Combination Index (CI) values generated by the software (Table 5).

TABLE 3
Concentration of agents for IC₅₀ value determination of single agents
in combination against Triple negative cell lines

| | | Top Concentration |
|---|---|---|
| Cell Line | Test Agent | (μM) |
| MDA-MB-231 | eribulin | 0.001 |
| | entinostat | 1 |
| | eribulin/entinostat | 0.0005/0.5 |
| HCC70 | eribulin | 0.002 |
| | entinostat | 2.5 |
| | eribulin/entinostat | 0.001/1.25 |
| HCC-1806 | eribulin | 0.001 |
| | entinostat | 3 |
| | eribulin/entinostat | 0.0005/1.5 |
| BT-549 | eribulin | 0.002 |
| | entinostat | 5 |
| | eribulin/entinostat | 0.001/2.5 |
| Hs578t | eribulin | 0.002 |
| | entinostat | 4 |
| | eribulin/entinostat | 0.001/2 |
| MDA-MB-436 | eribulin | 0.01 |
| | entinostat | 2 |
| | eribulin/entinostat | 0.005/1 |

TABLE 4
Concentration of agents for IC₅₀ value determination of single agents
in combination against Her2⁻ and ER⁺ cell lines

| | | Top Concentration |
|---|---|---|
| Cell Line | Test Agent | (μM) |
| MCF-7 | eribulin | 0.002 |
| | entinostat | 3 |
| | eribulin/entinostat | 0.001/1.5 |
| | eribulin/fulvestrant | 0.002/0.1 |
| | entinostat/fulvestrant | 3/0.1 |
| | eribulin/entinostat/fulvestrant | 0.001/1.5/0.1 |
| ZR-75-1 | eribulin | 0.025 |
| | entinostat | 30 |
| | eribulin/entinostat | 0.0125/15 |
| | eribulin/fulvestrant | 0.025/0.1 |
| | entinostat/fulvestrant | 30/0.1 |
| | eribulin/entinostat/fulvestrant | 0.0125/15/0.1 |
| T-47D | eribulin | 0.005 |
| | entinostat | 5 |
| | eribulin/entinostat | 0.0025/2.5 |
| | eribulin/fulvestrant | 0.005/0.1 |
| | entinostat/fulvestrant | 6/0.1 |
| | eribulin/entinostat/fulvestrant | 0.0025/3/0.1 |
| MDA-MB-134VI | eribulin | 0.08 |
| | entinostat | 4 |
| | eribulin/entinostat | 0.04/2 |
| | eribulin/fulvestrant | 0.08/0.1 |
| | entinostat/fulvestrant | 2/0.1 |
| | eribulin/entinostat/fulvestrant | 0.04/2/0.1 |
| MDA-MB-175VII | eribulin | 0.04 |
| | entinostat | 1.75 |
| | eribulin/entinostat | 0.002/0.875 |
| | eribulin/fulvestrant | 0.004/0.1 |
| | entinostat/fulvestrant | 1.75/0.1 |
| | eribulin/entinostat/fulvestrant | 0.002/0.875/0.1 |
| MDA-MB-415 | eribulin | 0.0025 |
| | entinostat | 0.125 |
| | eribulin/entinostat | 0.00125/0.0625 |
| | eribulin/fulvestrant | 0.0025/0.1 |
| | entinostat/fulvestrant | 0.25/0.1 |
| | eribulin/entinostat/fulvestrant | 0.00125/0.125/0.1 |

TABLE 5
Scales for description of Combination Index (CI)

| Range of Combination Index (CI) | Description |
|---|---|
| <0.5 | Strong synergy |
| 0.5-0.79 | Moderate synergy |
| 0.8-1.2 | Additivity |
| 1.21-1.5 | Moderate antagonism |
| >1.5 | Strong antagonism |

### Summary of Results

The inhibitory activity of eribulin and entinostat, alone and in combination, was evaluated against a panel of 12 human breast cancer cell lines as described in Table 1. Results for Experiments 1 and 2, IC₅₀ determination for single agents, are shown in Table 6. Overall, the results show that eribulin was more potent than entinostat in all cell lines Additionally, although the results should be more reliable using the narrow range dilutions, the values obtained from the wide range were not different from the results with the narrow range.

TABLE 6
Single Agent IC₅₀ Value Determination

| Cell Line | Test Agent | Wide range IC₅₀ | | Narrow range IC₅₀ | |
|---|---|---|---|---|---|
| MCF-7 | eribulin | 0.22 | nM | 0.37 | nM |
| | entinostat | 0.54 | μM | 0.57 | μM |
| T-47D | eribulin | nd | | 1.15 | nM |
| | entinostat | 1.60 | μM | 1.78 | μM |
| ZR-75-1 | eribulin | nd | | nd | |
| | entinostat | nd | | 4.95 | μM |
| MDA-MB-134VI | eribulin | 0.62 | nM | 0.88 | nM |
| | entinostat | 0.39 | μM | 0.80 | μM |
| MDA-MB-175VII | eribulin | 0.55 | nM | 1.09 | nM |
| | entinostat | 0.21 | μM | 1.17 | μM |
| MDA-MB-415 | eribulin | 0.64 | nM | 0.57 | nM |
| | entinostat | 92.14 | nM | 0.14 | μM |
| MDA-MB-231 | eribulin | 0.26 | nM | 0.52 | nM |
| | entinostat | 0.61 | μM | 0.94 | μM |
| HCC70 | eribulin | 0.35 | nM | nd | |
| | entinostat | 1.45 | μM | 1.11 | μM |
| HCC1806 | eribulin | 0.25 | nM | 0.30 | nM |
| | entinostat | 0.55 | μM | 1.22 | μM |
| BT-549 | eribulin | 0.23 | nM | 0.39 | nM |
| | entinostat | 1.25 | μM | 1.50 | μM |
| Hs578t | eribulin | 0.39 | nM | 0.67 | nM |
| | entinostat | 1.10 | μM | 1.40 | μM |
| MDA-MB-436 | eribulin | 2.82 | nM | 3.20 | nM |
| | entinostat | 0.81 | μM | 1.37 | μM |

nd, not determined (analysis could not be performed due to the biphasic shape of the inhibition curves).

The combination determination was carried out as previously described. Eribulin and entinostat were tested at concentrations according to Table 3 and Table 4 (above). Additionally, for Her-2⁻/ER⁺ cell lines, combinations were also carried out with a constant dose of 0.1 μM of fulvestrant across all dilutions. The CI values determined from the experimental Fa values for triple negative and Her-2⁻/ER⁺ breast cell lines are shown in Table 7 and Table 8, respectively. Of particular note, the experimental CI values for the combination of eribulin and entinostat in the triple negative breast cell lines listed in Table 7 show additivity based on ED₅₀ in HCC1806 and (VIDA-M8-436 cells, and additivity based on average ED_{50/75/90} in BT-549 and MDA-MB-231 cells. Moderate synergy was shown based on both ED₅₀ and ED_{50/75/90} in Hs578t cells. For the Her2−/ER+ breast cancer cell lines listed in Table 8, additivity was shown based on both ED₅₀ and ED_{50/75/90} in MCF-7 cells (+fulvestrant), MDA-MB-134VI cells (-fulvestrant), T-470 Cells (+/−fulvestrant), and MDA-MB-415 cells (−fulvestrant). Additivity based on ED₅₀ and moderate synergy based on ED_{50/75/90} was found in MDA-MB-415 cells (+fulvestrant). Moderate synergy was found based on both ED₅₀ and ED50/75/90 in MCF-7 cells (−fulvestrant).

TABLE 7
Calculated CI values for combination of compounds against
selected Triple Negative breast cell lines

| | | Calculated CI values | | | |
|---|---|---|---|---|---|
| | | | | | Average |
| Cell Line | Agents | ED₅₀ | ED₇₅ | ED₉₀ | (ED_{50/75/90}) |
| BT-549 | eribulin + | 1.47 | 0.99 | 0.86 | 1.10 |
| | entinostat | | | | |
| HCC1806 | eribulin + | 1.06 | 1.52 | 2.31 | 1.63 |
| | entinostat | | | | |
| HCC70 | eribulin + | nd | nd | nd | nd |
| | entinostat | | | | |
| Hs578t | eribulin + | 0.62 | 0.65 | 0.72 | 0.66 |
| | entinostat | | | | |
| MDA-MB-231 | eribulin + | 1.23 | 1.01 | 0.84 | 1.03 |
| | entinostat | | | | |
| MDA-MB-436 | eribulin + | 1.09 | 1.30 | 1.54 | 1.31 |
| | entinostat | | | | |

ED, effect dose.
nd, not determined (analysis could not be performed due to the biphasic shape of the inhibition curves)

TABLE 8
Calculated CI values for combination of compounds against
selected Her2⁻ and ER⁺ breast cell lines

| | | Calculated CI values | | | |
|---|---|---|---|---|---|
| | | | | | Average |
| Cell Line | Agents | ED₅₀ | ED₇₅ | ED₉₀ | (ED_{50/75/90}) |
| MCF-7 | eribulin + | 0.52 | 0.57 | 0.64 | 0.57 |
| | entinostat | | | | |
| | eribulin + | 0.86 | 0.78 | 0.77 | 0.80 |
| | entinostat + | | | | |
| | fulvestrant | | | | |
| MDA-MB- | eribulin + | 1.14 | 0.66 | 0.63 | 0.81 |
| 134VI | entinostat | | | | |
| | eribulin + | 1.50 | 3.18 | 9.43 | 4.70 |
| | entinostat + | | | | |
| | fulvestrant | | | | |
| MDA-MB- | eribulin + | 1.65 | 2.43 | 4.07 | 2.72 |
| 175VII | entinostat | | | | |
| | eribulin + | 1.32 | 2.20 | 4.10 | 2.54 |
| | entinostat + | | | | |
| | fulvestrant | | | | |
| T-47D | eribulin + | 1.13 | 1.00 | 1.04 | 1.06 |
| | entinostat | | | | |
| | eribulin + | 1.15 | 1.09 | 1.10 | 1.11 |
| | entinostat + | | | | |
| | fulvestrant | | | | |
| ZR-75-1 | eribulin + | nd | nd | nd | nd |
| | entinostat | | | | |
| | eribulin + | nd | nd | nd | nd |
| | entinostat + | | | | |
| | fulvestrant | | | | |
| MDA-ME-415 | eribulin + | 1.19 | 0.77 | 0.52 | 0.83 |
| | entinostat | | | | |
| | eribulin + | 0.85 | 0.41 | 0.29 | 0.52 |
| | entinostat + | | | | |
| | fulvestrant | | | | |

ED, effect dose.
nd, not determined (analysis could not be performed due to the biphasic shape of the inhibition curves).

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form. Other embodiments are within the scope of the following claims.

What is claimed is:

1. A method for treating a subject having or at risk of developing cancer, the method comprising administering to the subject (a) eribulin, or a pharmaceutically acceptable salt thereof, and (b) a histone deacetylase (HDAC) inhibitor.

2. The method of claim 1, wherein the pharmaceutically acceptable salt of eribulin is eribulin mesylate.

3. The method of claim 1, wherein the HDAC inhibitor is selected from the group consisting of a hydroxamic acid derivative, a carboxylic acid derivative, a benzamide derivative, a cyclic peptide, and an epoxyketone.

4. The method of claim 1, wherein the HDAC inhibitor is selected from the group consisting of trichostatin A, vorinostat, panobinostat, belinostat, givinostat, practinostat, quisinostat, abexinostat, CHR-3996, AR-42, valproate, butyrate, entinostat, entinostat polymorph B, mocetinostat, chidamide, apicidin, romidepsin, and trapoxins.

5. (canceled)

6. The method of claim 1, wherein the method consists of administering to the subject (a) eribulin mesylate and (b) the HDAC inhibitor.

7. (canceled)

8. The method of claim 1, wherein
(a) and (b) are administered substantially simultaneously;
(a) is administered first, followed by administration of (b);
(b) is administered first, followed by administration of (a);
(a) and (b) are administered substantially simultaneously, followed by administration of (a); or
(a) and (b) are administered substantially simultaneously, followed by administration of (b).

9-11. (canceled)

12. The method of claim 1, wherein the subject is a human.

13. The method of claim 1, wherein the subject is diagnosed with cancer, in treatment for cancer, or in post-therapy recovery from cancer.

14. The method of claim 1, wherein the cancer is a primary tumor, is locally advanced, or is metastatic.

15. The method of claim 1, wherein the cancer is selected from the group consisting of breast cancer, sarcomas, endometrial cancer, ovarian cancer, prostate cancer, leukemia, lymphoma, lung cancer, neuroendocrine tumors, pheochromocytoma, and thyroid cancer.

16. The method of claim 15, wherein said cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

17. The method of claim 15, wherein said cancer is a sarcoma selected from the group consisting of angiosarcoma, hemangiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leiomyosarcoma, and neurofibrosarcoma.

18. The method of claim 1, wherein the cancer is selected from the group consisting of stomach cancer, colon cancer, liver cancer, renal cancer, colorectal cancer, pancreatic cancer, cervical cancer, anal cancer, vulvar cancer, penile cancer, vaginal cancer, testicular cancer, pelvic cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, melanoma, a cancer of the central nervous system, a cancer of the respiratory system, and a cancer of the urinary system.

19. The method of claim 1, wherein the cancer is selected from the group consisting of B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), erythroleukemia, basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastic carcinoma, adrenocortical carcinoma, cholangiocarcinoma, Merkel cell carcinoma, ductal carcinoma in situ (DCIS), invasive ductal carcinoma, hepatoblastoma, medulloblastoma, nephroblastoma, neuroblastoma, pancreatoblastoma, pleuropulmonary blastoma, retinoblastoma, glioblastoma multiforme, Hodgkin's lymphoma, non-Hodgkin's lymphoma, Burkitt lymphoma, follicular lymphoma, thymoma, multiple myeloma, plasmacytoma, localized myeloma, extramedullary myeloma, superficial spreading melanoma, nodular melanoma, lentigno maligna melanoma, acral lentiginous melanoma, amelanotic melanoma, ganglioneuroma, Pacinian neuroma, acoustic neuroma, astrocytoma, oligoastrocytoma, ependymoma, brainstem glioma, optic nerve glioma, oligoastrocytoma, pheochromocytoma, meningioma, malignant mesothelioma, and a virally induced cancer.

20. The method of claim 1, wherein the cancer is a hormone responsive cancer.

21. The method of claim 1, wherein said subject is an adult patient.

22. The method of claim 1, wherein said subject is a pediatric patient.

23-30. (canceled)

31. A method for decreasing the size of a tumor in a subject, the method comprising administering to the subject (a) eribulin, or a pharmaceutically acceptable salt thereof, and (b) an HDAC inhibitor.

32-33. (canceled)

34. The method of claim 1, further comprising administering to the subject one or more additional therapeutic agents, which optionally are selected from anti-hormonal agents (e.g., fulvestrant, tamoxifen, toremifene, or aromatase inhibitors), immunomodulatory agents (e.g., antibodies or vaccines), chemotherapeutic/antitumor agents, antibacterial agents, anti-emetics, and anti-inflammatory agents.

35. A kit for use in treating cancer or decreasing tumor size in a subject, the kit comprising (a) eribulin, or a pharmaceutically acceptable salt thereof, and (b) an HDAC inhibitor, optionally in dosage form.

36-39. (canceled)
